(19) United States
(12) Patent Application Publication
(10) Pub. No.: US 2012/0209223 A1
(43) Pub. Date: Aug. 16, 2012

(54) TRANSDERMAL THERAPEUTIC SYSTEM FOR ADMINISTRATION OF FENTANYL OR AN ANALOG THEREOF

(75) Inventors: Nouha Salman, Muenchen (DE); Bjoern Schurad, Muenchen (DE); Ingo Teutsch, Muenchen (DE)

(73) Assignee: ACINO AG, Miesbach (DE)

(21) Appl. No.: 13/496,039

(22) PCT Filed: Sep. 14, 2010
(86) PCT No.: PCT/EP2010/063433

§ 371 Date: Apr. 24, 2012

(30) Foreign Application Priority Data
Sep. 14, 2009 (EP) 09170223.3

(51) Int. Cl. A61M 35/00; A61K 31/445; B32B 27/06; A61K 9/00

(52) U.S. Cl. 604/307; 424/400; 514/329; 156/278

(57) ABSTRACT

Disclosed is a transdermal therapeutic system for administering an active ingredient through the skin comprising:
a) a back layer,
b) a reservoir on the back layer comprising
   b1) a first layer containing active ingredient, at least one gel former, at least one plasticizer, and a first polyisobutylene; and
   b2) a second layer containing active ingredient, at least one gel former, at least one plasticizer, and a second polyisobutylene,
      wherein the first polyisobutylene is different from the second polyisobutylene, wherein at least the first layer contains undissolved active ingredient in the form of active ingredient particles; and wherein the active ingredient is fentanyl or an analogue of the fentanyl.

Object of the present application is a system for the transdermal administration of fentanyl or an analogue thereof for therapeutic purposes. The TTS is characterized by a low content of active ingredient and a small patch size.

Fentanyl and its analogues, in particular alfentanil, carfentanil, lofentanil, remifentanil, sufentanil, trefentanil, and related compounds are potent synthetic opiates. Fentanyl and its analogues are highly efficacious and are rapidly metabolized. A problem with fentanyl is its relatively narrow therapeutic index. When the threshold values are exceeded undesired side effects occur, in particular impairment of respiration what can—unless suitable countermeasures are taken—cause death. The active ingredients are relatively expensive and there is a high risk of abuse. That's why fentanyl patches on the one hand have to ensure a very precisely controlled release of the active ingredient and on the other hand the product should be designed such that the active ingredient cannot be removed easily out of it for purposes of abuse.

Usually, a transdermal patch is a small adherent bandage containing the active ingredient to be delivered. These bandages can have various forms and sizes. The simplest type is an adhesive monolith comprising an active ingredient stock (reservoir) on a carrier. Usually, the reservoir is formed of the active ingredient in a pharmaceutically acceptable pressure-sensitive adhesive. However, it can also be formed of a non-adherent material the skin-contacting surface of which is provided with a thin layer of a suitable adhesive.

More complex patches are multiple laminates or patches having an active ingredient stock (which can optionally be solved in a liquid) wherein a membrane controlling the release of the active ingredient can be arranged between the reservoir and the skin-contacting adhesive. This membrane is for the control and optionally reduction of the effects of variations of the skin permeability by lowering the rate of delivery in vitro and in vivo of the active ingredient from the patch.

The reservoir of the transdermal patches can contain the active ingredient either completely dissolved in the stock or it can contain an excess of undissolved active ingredient beyond its saturation concentration (depot patch). However, the presence of undissolved active ingredient or other constituents in a patch can cause stability and other problems in storage as well as in use. Also a difficulty is that it has to be ensured that the active ingredient dissolves sufficiently fast enough from the solid depot additionally to replace the delivered active ingredient. In the state of the art, active ingredient patches the reservoir of which has solid active ingredient particles are often considered to be detrimental.

Various transdermal patches for the administration of fentanyl are known from the state of the art. WO 02/074286 describes a transdermal patch having a reservoir containing fentanyl wherein the reservoir has a polymeric composition, preferably polyacrylate in a uniform phase state being free of undissolved active ingredient. Here, a supersaturation should explicitly be avoided.

There are many experiments to prepare fentanyl patches also on the basis of a matrix layer of polyisobutylene. First, such experiments are already described in the basic patent regarding fentanyl patches U.S. Pat. No. 4,588,580. This publication discloses a transdermal therapeutic system with a polyisobutylene matrix and mineral oil containing a 2% load of fentanyl as undissolved solid. However, in practice the system has disadvantages and in the period following the development departed from polyisobutylene matrices and if any polyisobutylene matrices were used the attempt was made to completely solve the active ingredient in the polyisobutylene matrix.

A transdermal therapeutic system with a polyisobutylene matrix is described in Roy et al., Journal of Pharmaceutical Sciences, Vol. 85, No. 5, May 1996, pp 491 to 495. It is shown that with concentrations of fentanyl in the polyisobutylene matrix of more than 4% active ingredient is precipitating and this Roy et al. obviously considered negative. For patches with a low active ingredient loading Roy et al. suggest a fentanyl polysilicone patch and not polyisobutylene patches.

In EP 1 625 854, US 2007/0009588, and US 2006/0013865 corresponding polyisobutylene matrices are suggested in case of which it is carefully to attend that the active ingredient is present completely dissolved in the polyisobutylene matrix. Occurring of crystals in the matrix is considered to be negative.

DE 198 37 902 discloses transdermal therapeutic systems on the basis of polyisobutylene particularly suitable for administering clonidine, however, among the active ingredients mentioned there fentanyl is also found. Examples of fentanyl patches are not found in this printed matter, an indication that in the patches disclosed there the active ingredient should be present as a solid is also not found in this printed matter. The printed matter does not contain in vivo studies on the release of the active ingredient from the patches. The polyisobutylene layer of these patches contains at least 5% by weight of a filler.

The not pre-published European Patent application with the application number 08155167.3 describes transdermal therapeutic systems for administering fentanyl or an analogue thereof wherein a polyisobutylene layer containing the active ingredient and having a content of gel former of at most 4% by weight is applied on the back layer. The transdermal therapeutic systems described there may have applied also an adhesive layer to the polyisobutylene layer, however said adhesive layer does not contain fentanyl, and the composition of the adhesive layer is not specified. With these patches an extended and more uniform release should be achieved than with the known fentanyl patches.

US 2008/0175890 A1 refers to sufentanyl patches designed to provide the highest possible flow of sufentanyl through the skin without using penetration enhancers. The patches on the basis of PIB disclosed there contain neither plasticizers nor gel formers. The patches disclosed there do not show the properties that are of advantage for a commercial patch such as good adhesive strength in conjunction with a long-lasting, slow release of the active ingredient with a very small patch.

EP 0 272 987 also discloses a patch for administering active ingredients, wherein a specifically designed support layer is considered relevant. In one example of the printed matter a fentanyl patch is disclosed that contains two active ingredient layers, wherein one of the active ingredient layers represents a polyisobutylene layer and the other represents a polydimethyl siloxane layer. The printed matter discloses that the active ingredient flow from a PIB layer is substantially lower than from a polydimethyl siloxane layer.

WO 01/64149 discloses active ingredient patches with a single active ingredient layer, wherein the active ingredient is partially dissolved.

Currently, there is no market product yet that is a fentanyl patch on the basis of polyisobutylene.

The known fentanyl patches on the market are typically matrix patches on the basis of polyacrylates, they all have a high content of active ingredient, and a relatively large area of not less than 8 cm². In the application to the skin of the patient a larger area is considered as a disadvantage. Due to the high prices of the active ingredients and the potential for abuse it would thus be advantageous to develop patches that on the one hand have a low content of active ingredient and on the other hand have a minimal residual content of active ingredient.

Examples of fentanyl patches customary in the market are DUROGESIC® SMAT having 4.2 mg of active ingredient at a patch size of 10.5 cm². Less active ingredient and a smaller patch size has the market product MATRIFEN® a patch on the basis of polysilicone that has a content of active ingredient of 2.75 mg at a patch size of 8.4 cm². However, there is a need for a patch that has possibly an even lower content of active ingredient at the same or even a smaller patch size than the known patches. The patches should have an adhesive capacity as good as possible, i.e. adhere to the skin over the intended period of use of typically three days or even more, nevertheless be easily removable again and without pain. Moreover, they should provide the same or at least a quite similar plasma level curve such as the known market products, and in particular the market product DUROGESIC® SMAT. In particular, they should preferably be substantially bioequivalent to the product DUROGESIC® SMAT, wherein for the definition of the term “bioequivalency” it is pointed to WO 02/074286 to the relevant disclosure of which it is explicitly referred.

Regarding the prior art, there remains in particular the object to provide a transdermal therapeutic system for administering fentanyl or an analogue thereof through the skin that does not show the problems of the prior art. In particular, the patch should be able to uniformly release fentanyl or an analogue thereof over a long period of time in the amount required for pain control. The plasma level should remain constant over a period of time as long as possible, but preferably over a period of about 30 hours after the administration to about 72 hours of the administration such that it substantially corresponds to the plasma level that is reached by the market product DUROGESIC® SMAT. Preferred embodiments are those wherein the patch is designed for the administration every three days, every four days, every five days, every six days, or every seven days. It is thus preferred that the plasma level is substantially constant until for example there arises a three-day patch, a four-day patch, a five-day patch, a six-day, or a seven-day patch. According to the invention a three-day patch is particularly preferred with correspondence to the time of administration of the product DUROGESIC® SMAT.

In addition, the content of active ingredient (at the same delivery rate) compared to the patches customary in the market of the corresponding active ingredient shall be noticeably reduced and the patch shall have a smaller area than patches customary in the market of the corresponding active ingredient (again of course at the same delivery rate).

Object of the present invention is thus a transdermal therapeutic system for administering an active ingredient through the skin comprising:

a) a back layer,

b) a reservoir on the back layer comprising
b1) a first layer containing active ingredient, at least one gel former, at least one plasticizer, and a first polyisobutylene; and
b2) a second layer containing active ingredient, at least one gel former, at least one plasticizer, and a second polyisobutylene,

wherein the first polyisobutylene is different from the second polyisobutylene,

wherein at least the first layer contains undissolved active ingredient in the form of active ingredient particles; and

wherein the active ingredient is fentanyl or an analogue of the fentanyl.

In addition to fentanyl according to the invention analogues of the fentanyl are preferred such as alfentanil, lofentanil, remifentanil or sufentanil wherein it is especially preferred that the active ingredient is fentanyl or sufentanil, in particular fentanyl. In the following the invention is explained substantially with regard to the fentanyl. However, the embodiments apply correspondingly also to the analogues of the fentanyl.

Preferably, with a patch according to the invention it is possible to cause analgesia in a patient over the intended period of use (particularly preferred of three days), the exploitation of active ingredient being very high. Particularly preferred, in the patches according to the invention the ratio of area to content of active ingredient is in the range of from 2 to 5, more preferred from 2 to 4, in particular from 2.2 to 3.8.

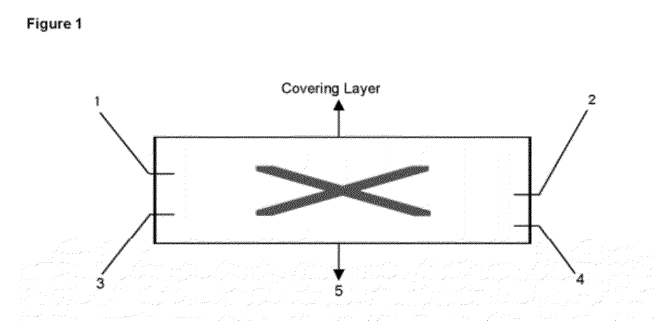

The construction of a preferred transdermal therapeutic system in a cross-section is represented in FIG. 1. In the preferred transdermal therapeutic system of FIG. 1 the transdermal therapeutic system consists of a back layer 1, a reservoir consisting of a first layer 2 and a second layer 4 as well as a stripping layer 5. The patch may also contain a membrane 3 (optional), which is however preferably not present. Furthermore, also a covering layer may be present that rests substantially loose on the back layer and adheres thereto for example by electrostatic forces. The covering layer is known and serves to facilitate the removal of the patch from the package. It is not to be confused with the stripping layer 5. Both, the membrane 3 and the covering layer and the stripping layer 5 are optional, wherein typically the stripping layer 5 will be present in the patch. In FIG. 1 the asterisks in the first layer 2 symbolize the undissolved active ingredient present in this layer.

According to the invention this construction of the transdermal therapeutic system is most preferred. However, other embodiments are also possible, wherein in addition to the first layer and the second layer still further layers are provided (e.g., back layers without active ingredient, further reservoir layers etc.). These embodiments will not be described in detail in the following, but may be readily prepared by the person skilled in the art due to the disclosure of the present application. Thus, the following embodiments refer to the preferred embodiment of the transdermal therapeutic system according to the invention with exactly two active ingredient-containing layers and optionally a membrane wherein no further layers are present. However, the explanations analogically apply also to further not represented embodiments with additional layers that still may optionally also contain active ingredient.

The back layer 1 is located at the end of the patch that is in use opposite to the skin. At the side of the back layer 1 that in use is faced to the human skin there is located the reservoir comprising a first layer 2 and a second layer 4. The first layer 2 contacting the back layer 1 preferably has a higher content of active ingredient than the second layer 4 facing the skin.

According to the invention it is preferred that the adhesive capacity of the second layer 4 is higher than the adhesive capacity of the first layer 2 so that the second layer also takes on the function of a back layer.

According to the invention it is particularly preferred that the adhesive capacity of the second layer is such that the transdermal therapeutic system over the intended period of action safely sticks to the skin and can be removed from the skin without skin damages or irritations.

At least in the first layer 2 a dissolved and undissolved active ingredient is present in the form of active ingredient particles that will be discussed in more detail below.

When reference is made in the context of the present application to the adhesive capacity the measurement of the adhesive capacity is made according to the corresponding German Standards Association (DIN) regulations. Unless, according to the invention, it depends on the absolute adhesive capacity of the layers, but only on the relative adhesive capacity of the first layer to the second layer also another common method for determining the adhesive capacity may be used that differs from the DIN method as long as the adhesive capacity of the first layer and the second layer are determined according to the same method.

On the second layer 4 there is preferably also a stripping layer 5 that before using the transdermal therapeutic system is withdrawn.

The first and second layers preferably have the same area in the patch so that none of the layers protrudes beyond the other.

In a further embodiment an additional membrane 3 is provided in the transdermal therapeutic system between the first and the second layers that controls the release of the active ingredient. The main purpose of said membrane is to reduce the in vivo and in vitro release rate of the active ingredient from the patch. In this way, differences in the permeability for the active ingredient through the skin may be balanced. Preferably, the membrane is a microporous membrane.

Suitable membranes are known in the state of the art. In a preferred embodiment the membrane can contain or may be composed of polypropylene or polyethylene vinylacetate. An especially preferred material for the membrane is a microporous polypropylene film.

The thickness of the membrane is not particularly restricted and may e.g., be in the range of from 10 μm to 100 μm, preferred less than 50 μm, e.g., about 25 μm. The pore size is preferably in the range of from 0.001 to 0.025 μm², e.g., in the range of from 0.002 to 0.011 μm², particularly about 0.005 μm². Also the shape of the pores is not particularly restricted, a rectangular shape is preferred.

Hence, a typical example of a suitable membrane is a microporous polypropylene film having a thickness of about 25 μm and a pore size of about 0.12 μm×0.04 μm, as marketed under the trade name Celgard 2400 of Celgard LLC, Charlotte, USA.

Preferably the patch according to the invention has no membrane.

On the second layer 4 there is a stripping layer (release liner) indicated in FIG. 1 with the number 5. Said stripping layer is preferably made of a polymeric material that optionally may also be metalized. Examples of preferably employed polymeric materials are polyurethane, polyvinyl acetate, polyvinylidene chloride, polypropylene, polycarbonate, polystyrene, polyethylene, polyethylene terephthalate, polybutylene terephthalate as well as optionally paper surface-coated with corresponding polymers. Preferably, it is a stripping layer that is fluorinated or siliconized on one or both sides. Particularly preferred are commercial fluorinated or siliconized polyester films such as the trade products siliconized on one side Primeliner 100 μm, Perlosic LF 75 μm (Loparex, NL and Perlen Converting AG, Switzerland), more preferred are the trade products fluorinated on one side Scotchpak 1022 or Scotchpak 9742 (3M).

It is essential according to the invention that both the first layer and the second layer contain the active ingredient, preferably fentanyl. According to the invention it is preferred that the concentration of the active ingredient in the first layer is higher than in the second layer. According to the invention a part of the fentanyl in the first layer is present in an undissolved form so that the concentration of the dissolved fentanyl in the first layer corresponds to the saturation solubility of the fentanyl in the first layer, but the total concentration of fentanyl in the first layer (dissolved +undissolved) is above the saturation solubility of the fentanyl in the first layer.

Preferably, the total concentration of the fentanyl in the first layer (dissolved constituent of the fentanyl and undissolved constituent of the fentanyl) is in the range of 5% to 15% by weight, preferably in the range of 8% to 12% by weight, and more preferred in the range of 9% to 11% by weight. The unit percentage by weight refers to the total weight of the first layer including the active ingredient and all the other constituents.

In the second layer the concentration of the fentanyl is preferably below the saturation solubility of the fentanyl in the second layer so that all of the fentanyl in the second layer is preferably present in a dissolved form. However, according to the invention it is also possible that in both layers, in the first layer and in the second layer, the fentanyl is partially present in the form of crystals.

The concentration of the fentanyl in the second layer is preferably 0.5% by weight to 3% by weight, more preferred 1 to 2.5% by weight, and particularly 1% by weight to 2% by weight. The indications above refer to the total weight of the second layer including the active ingredient and all of the remaining constituents.

Where in the present application reference is made to the saturation solubility or the saturation concentration of fentanyl, respectively, these indications refer to a temperature of 25° C., and the saturation solubility is determined as follows: a series of patches with increasing fentanyl concentrations is prepared and stored at 25° C. The patches are observed over a period of six months. The highest concentration at which no active ingredient has crystallized out after six months (or precipitated as a solid, respectively) represents the lower limit of the saturation solubility. The lowest concentration at which active ingredient has crystallized out after six months (or precipitated as a solid, respectively) represents the upper limit of the saturation solubility. The saturation solubility is then within the range formed by the two limits. The accuracy of the measuring method can be increased by reduction of the distances between the concentrations. The observation is optically (with the eye).

The saturation solubility of the fentanyl in both layers can be adjusted in a known manner, e.g. by adding substances to the layers that influence the saturation solubility or by selection of corresponding matrix polymers. According to the invention it is particularly preferred that the adjustment of the saturation solubility takes place by the variation of the plasticizer or the concentration of the plasticizer, respectively, in the layer.

According to the invention it is preferred that the fentanyl concentration in the second layer is lower than the fentanyl concentration in the first layer. Preferably, the ratio of fentanyl in the first layer to fentanyl in the second layer (weight basis) is in the range of 4 to 12, more preferred in the range of 6 to 10, and particularly in the range of 7 to 9.

The concentration of the active ingredient in the whole reservoir, that is both in the first layer and in the second layer and optionally further active ingredient-containing layers of the reservoir, is preferably 3% by weight to 20% by weight, more preferred 4% by weight to 15% by weight, in particular 5% by weight to 10% by weight, wherein the weight percentages refer to the total weight of all active ingredient-containing layers in the reservoir including the active ingredient and all the other constituents of the layer.

This preferably results in area weights in the range of 20 to 100 g/m², more preferred 25 to 80 g/m², in particular in the range of 30 to 70 g/m². The reservoir has preferably a thickness (dry thickness) in the range of 20 to 400 μm, more preferred in the range of 30 to 200 μm, in particular in the range of 40 to 100 μm (all layers including the optionally present membrane).

It is understood that in the transdermal therapeutic systems according to the invention all of the active ingredient-containing layers are constituents of the reservoir, that is no active ingredient-containing layers are located outside of the reservoir.

The plasticizer is a basically known compound employed in the state of the art in transdermal therapeutic systems as plasticizer. Preferably, the plasticizer is also designed to enhance the penetration of the active ingredient through the skin and in an especially preferred embodiment it regulates the solubility of the active ingredient in the reservoir such that a certain content of active ingredient is maintained in solution. Thus, the plasticizer is present in both layers, the first layer and the second layer, as defined above. It is preferred that the plasticizer is the same compound in both layers, it is however also possible that in the first layer a plasticizer other than that of the second layer is present, in particular, if desired that the plasticizer adjusts the solubility of the active ingredient in the first layer different to that in the second layer.

Preferably, the plasticizer is mineral oil, linseed oil, octyl palmitate, squalene, squalane, silicone oil, isobutyl myristate, isostearyl alcohol, or oleyl alcohol wherein mineral oils are the preferred plasticizers. These oils which are also referred to as thin paraffins are colorless clear hydrocarbons. They are obtained from the petroleum distillation fractions boiling above 300° C. and are liberated from solid hydrocarbons by cooling. They are refined by extraction with solvents as well as by treatment with bleaching earths and/or sulfuric acid. Suitable mineral oils are both chemically and biologically stable and prevent bacterial growth. By suitable fractionation mineral oils can be obtained that are liquid around body temperature, i.e. at about 35 to 37° C., and are solid at lower temperatures, especially at temperatures below 20° C. Preferred is to choose a mineral oil having a liquefaction point of about 30-35° C.

Preferably, the percentage of plasticizer in the first layer is higher than in the second layer.

Preferably, the plasticizer is present in the first layer in an amount ranging from 10 to 60% by weight, more preferably from 25 to 50% by weight, in particular ranging from 30 to 40% by weight (based on the total weight of the first layer).

Preferably, the plasticizer is present in the second layer in an amount ranging from 1 to 15% by weight, more preferably from 2 to 10% by weight, in particular ranging from 3 to 8% by weight, e.g. about 5% by weight (each based on the total weight of the second layer).

In the transdermal therapeutic system the reservoir has to contain an amount of fentanyl or an analogue thereof sufficient to induce analgesia in a human being and to maintain it for the desired period of time, preferably at least two days, more preferred at least three days (based on the point of administration of the patch). Preferably, the reservoir contains an amount of fentanyl or of an analogue thereof sufficient to induce analgesia and to maintain it for a period of at least three days, in particular three to seven days, in particular three days.

The layers of the patch according to the invention also contain a gel former. Preferably, this is a gel former with a particulate structure having on its surface a high concentration of polar groups. Said groups cause correspondingly high interfacial tensions towards the oils (plasticizer) which are partially compensated by agglomeration of the particles among themselves to gel skeletons. Accordingly, the greater the difference in polarity between the oils and the skeleton former surface the stronger are the gel skeletons. According to the invention it is preferred to employ as the gel former highly disperse silica or colloidal silica. The particle size is preferably in the nano-area and is e.g., in the range of 400 to 1500 nm, in particular in the range of 500 to 1000 nm. For example, colloidal silica is marketed under the designation Cab-O-Sil® and is a known thickener for mineral oil. Another example for a suitable gel former is bentonite. Also sodium carbomer known as gel former can be used. Preferably, the gel former is used in each layer in an amount of 0.1 to 4.0, more preferably 0.5 to 2.0% by weight, based on the total weight of the respective layer.

As pointed out with the plasticizer it is also preferred for the gel former that each layer, in particular the first and the second layer, has the same gel former. However, it is also possible that in each layer a different gel former is employed.

According to the invention it is essential that the polyisobutylene of the first layer (first polyisobutylene) is different from the polyisobutylenes of the second layer (second polyisobutylene). According to the invention the first polyisobutylene preferably has an other average molecular weight than the second polyisobutylene (in the context of this application reference is always made to the weight average molecular weight M_{w}, unless otherwise specified or obvious due to the context; the weight average molecular weight M_{w} is typically determined by GPC, as is known to the skilled person).

According to the invention the average molecular weight of the first polyisobutylene is preferably in the range of 100,000 to 10,000,000, and the average molecular weight of the second polyisobutylene is in the range of 15,000 to 5,000,000, wherein the average molecular weight of the second polyisobutylene is lower than the average molecular weight of the first polyisobutylene.

According to the invention the first polyisobutylene preferably consists of a mixture of at least two polyisobutylenes having different average molecular weights. That means that the molecular weight distribution of the first polyisobutylene has at least two peaks at different molecular weights.

That is, the first polyisobutylene is preferably a mixture of a polyisobutylene with a first weight average molecular weight and a polyisobutylene with a second weight average molecular weight, wherein the first weight average molecular weight is higher than the second weight average molecular weight. Particularly preferred in this embodiment is that the ratio of the polyisobutylene with the first weight average molecular weight to the polyisobutylene with the second weight average molecular weight is in the range of 1:0.1 to 1:10, more preferred in the range of 1:0.5 to 1:2, e.g. about 1:1.

According to the invention the second polyisobutylene preferably consists of a mixture of at least two polyisobutylenes having different average molecular weights. That means that the molecular weight distribution of the second polyisobutylene also has at least two peaks at different molecular weights.

That is, the second polyisobutylene is preferably a mixture of a polyisobutylene with a first weight average molecular weight and a polyisobutylene with a second weight average molecular weight, wherein the first weight average molecular weight is higher than the second weight average molecular weight. Particularly preferred in this embodiment is that the ratio of the polyisobutylene with the first weight average molecular weight to the polyisobutylene with the second weight average molecular weight is in the range of 1:1 to 1:100, more preferred in the range of 1:5 to 1:20, e.g. about 1:9 or 1:10.

According to the invention it is particularly preferred that both the first and the second polyisobutylene consist of a mixture of at least two, more preferred of two polyisobutylenes, one with a higher average molecular weight, one with a lower average molecular weight.

Preferably, one polyisobutylene of the mixtures (higher molecular polyisobutylene; polyisobutylene with a first weight average molecular weight) has an average molecular weight of 150,000 to 10,000,000, particularly preferred of 500,000 to 10,000,000, and the other polyisobutylene of the mixtures has a lower average molecular weight (lower molecular polyisobutylene; polyisobutylene with a second weight average molecular weight) in the range of 15,000 to 100,000, preferably 20,000 to 80,000. The polyisobutylene with the lower average molecular weight above all is responsible for the adhesiveness of the patch.

In one embodiment of the present invention the average molecular weight of the higher molecular polyisobutylene in the mixture of the first polyisobutylene is different from the average molecular weight of the higher molecular polyisobutylene in the mixture of the second polyisobutylene and/or the average molecular weight of the lower molecular polyisobutylene in the mixture of the first polyisobutylene is different from the average molecular weight of the lower molecular polyisobutylene in the mixture of the second polyisobutylene.

However, according to the invention it is preferred that the average molecular weight of the higher molecular polyisobutylene in the mixture of the first polyisobutylene and the mixture of the second polyisobutylene are substantially the same. According to the invention it is also preferred that also the average molecular weight of the lower molecular polyisobutylene in the mixture of the first polyisobutylene and in the mixture of the second polyisobutylene are substantially the same. The first polyisobutylene then differs from the second polyisobutylene in that the ratio of both polyisobutylenes (higher molecular polyisobutylene:lower molecular polyisobutylene and polyisobutylene with a first weight average molecular weight:polyisobutylene with a second weight average molecular weight, respectively) in the mixture of the first polyisobutylene differs from that in the mixture of the second polyisobutylene.

That is, the portion of the polyisobutylene with a high molecular weight in the mixture of the first polyisobutylene is different from that in the mixture of the second polyisobutylene, and accordingly the portion of the polyisobutylene with a lower molecular weight in the mixture of the first polyisobutylene is different from that in the mixture of the second polyisobutylene.

According to the invention “substantially the same” means that the respective values (for example of the average molecular weight) do not differ by more than 10%, based on the highest value. Preferably this is understood to mean that the values are the same within the measuring accuracy.

Preferably, in the mixture of the first polyisobutylene the ratio of the polyisobutylene with a higher molecular weight to the polyisobutylene with a lower molecular weight is in the range of 0.05:1 to 20:1, particularly preferred 0.5:1 to 2:1, in particular in the range of about 1:1.

In the mixture of the second polyisobutylene the portion of polyisobutylene with a lower molecular weight is typically higher than in the mixture of the first polyisobutylene, and in particular here the ratio of polyisobutylene with a lower molecular weight to the polyisobutylene with a higher molecular weight is preferably in the range of 1:1 to 30:1, in particular in the range of 2:1 to 15:1, for example about 9:1, that is that about nine times as much polyisobutylene with a lower molecular weight as polyisobutylene with a higher molecular weight is present (all indications are based on weight).

Based on the total weight of the first layer the portion of the first polyisobutylene is preferably 30 to 80% by weight, more preferred 40 to 65% by weight, in particular 50 to 60% by weight.

Based on the total weight of the second layer the portion of the second polyisobutylene is preferably 50 to 98% by weight, more preferred 60 to 95% by weight, in particular 80 to 95% by weight, e.g. about 93% by weight.

Polyisobutylenes with a certain weight average molecular weight are commercial and may be used as the first and the second polyisobutylene, respectively, in accordance to the invention. The mixtures of at least two polyisobutylenes with different molecular weights that are particularly preferred according to the invention may e.g. be prepared by mixing the commercial polyisobutylenes. For that, both commercial polyisobutylenes are dissolved in a suitable solvent, e.g. n-heptane, and mixed. Subsequently, the solvent can suitably be removed.

Examples of polyisobutylenes with a higher molecular weight (that is polyisobutylene with a first weight average molecular weight) are the commercial products Oppanol B80, B100, B150, and B200, preferably Oppanol B80 or B100, in particular Oppanol B100 (M_{w}=1,100,000), and examples of polyisobutylenes with a lower molecular weight (that is polyisobutylene with a second weight average molecular weight) are the commercial products Oppanol B10 SFN to B15 SFN, in particular Oppanol B10 SFN (M_{w}=36,000). An example of a preferred first polyisobutylene is a mixture of Oppanol B100 and Oppanol B10 SFN at a ratio of 1:1, and an example of a preferred second polyisobutylene is a mixture of Oppanol B100 and Oppanol B10 SFN at a ratio of 1:9.

Further preferred examples of the first and second polyisobutylene mixtures are as follows:

| | First Polyisobutylene | Second Polyisobutylene |
|---|---|---|
| | Mixture Oppanol B100/ | Mixture Oppanol B100/ |
| No. | Oppanol B10 SFN | Oppanol B10 SFN |
| 1 | 4:6 | 2:8 |
| 2 | 6:4 | 3:7 |
| 3 | 7:3 | 4:6 |
| 4 | 8:2 | 5:5 |
| 5 | 1:1 | 1:15 |

Also preferred is the use of Oppanol B12 SFN instead of Oppanol B10 SFN in the above mixtures.

Whenever in this description it is talked about a “total weight” this is always understood to mean the dry weight including all the constituents, that is the weight in the patch ready to use unless otherwise disclosed or obvious.

It is essential for the transdermal therapeutic systems according to the invention that at least the first layer contains as much active ingredient that a part of the active ingredient is present in an undissolved form, that is as active ingredient particle.

In the production of the patch according to the invention the active ingredient is preferably used in a micronized form having a mean particle size of 50 μm or less, preferably having a mean particle size of 20 μm or less. Also, in the first layer of the patch the active ingredient is present in a micronized form, however there can result minor deviations of the particle size by rearrangement reactions when storing the patch. However, also, within the patch the mean particle size of the active ingredient particles is preferably less than 100 μm, more preferred less than 50 μm, and in particular about 20 μm or less. For the production of the patch according to the invention there is preferably used micronized fentanyl having a mean particle size of 1 μm or more, more preferred 2 μm or more. Also, said mean particle sizes are preferably found in the finally produced patches. In the layers of the transdermal therapeutic system the particle size and the particle-size distribution of the active ingredient particles can be determined by conventional light microscopy. The evaluation is carried out with conventional computer programs (image processing systems) that as a rule are adapted to the microscopes used. Unless otherwise indicated or apparent the particle size refers to the particle diameter.

As the starting material for the micronized fentanyl there is used the commercially available fentanyl which is per se suitable for clinical application. Typically, such fentanyl shows a distribution of the particle size such that 100% of the particles are smaller than 2,500 μm. About 90% of the particles are smaller than about 1,000 μm, and 50% of the particles are smaller than about 100 μm.

According to the invention any known micronization process providing the desired particle size can be used. It is preferred to use fentanyl that was micronized by means of a conventional “jet mill”, e.g., a jet mill of the AS type by Hosokawa Alpine AG.

By the micronization process used in accordance to the invention the size of the fentanyl particles is preferably adjusted such that the mean particle size is in the above-mentioned ranges. It is also preferred that 100% of the particles are smaller than 50 μm, in particular smaller than 20 μm. Preferably, about 90% of the micronized particles are smaller than 12 μm, and about 50% of the particles are smaller than 6 μm.

There are various methods for determining the particle size and the particle-size distribution of the active ingredient, for example the light scattering method (laser diffractometry) as used in the devices of Malvern Instruments, e.g., the “Malvern MasterSizer X”, the mechanical sieve shaking method as used by FMC for determining the grain-size distribution of its AVICEL PH® products, or also “air jet” sieve analyses which can be performed with an ALPINA® “air jet” model 200.

Unless otherwise stated, the (average) particle sizes and particle-size distributions, respectively, are determined with the laser diffractometry method, for example with the Mastersizer 2000 from Malvern.

If the active ingredient is defined by the indication of the mean particle size and the particle-size distribution the micronized active ingredient used according to the invention has preferably an average particle size of 20 μm or less and it is preferred that the active ingredient has a grain-size distribution (particle-size distribution) wherein less than 10% of the particles have a size of 25 μm or more and less than 10% of the particles have a size of 1 μm or below. Further preferred is an active ingredient having an average particle size of 15 μm. Preferably, such an active ingredient has a grain-size distribution wherein less than 2% of the particles have a size of 20 μm or more and less than 50% of the particles have a size of 6 μm or below. The grain-size distribution for said active ingredient should be as narrow as possible.

On the side of the reservoir that is in use turned away from the human skin there is a back layer being in a preferred embodiment occlusive, i.e. ending. In a preferred embodiment such back layers can consist of polyolefines, in particular polyethylene, or of polyester as well as polyurethanes. Also, layers containing several different polymers arranged one upon the other may be employed advantageously. A particularly preferred material for the back layer is a polyolefine marketed by Mylan Technologies Inc. under the designation Mediflex®1000. Other suitable materials comprise cellophane, cellulose acetate, ethyl cellulose, plasticizer-containing vinyl acetate vinyl chloride copolymers, ethylene vinyl acetate copolymers, polyethylene terephthalate, nylon, polyethylene, polypropylene, polyvinylidene chloride, ethylene methacrylate copolymer, paper which optionally can be coated, textile fabrics, polyester films such as polyethylene terephthalate films. Particularly preferred are aluminum films and polymer metal composite materials. The thickness of the back layer is, e.g., 10 μm to 80 μm, e.g., about 55 μm nominal thickness as common in the state of the art.

On the back layer of the patch there is preferably a covering layer that shall in particular prevent that the patch sticks to the package in case small amounts of polyisobutylene or adhesive pass out. Preferably, the covering layer lies loose on the back layer and is kept by electrostatic forces. Such covering layers are known in the state of the art, e.g., from EP 1 097 090, which is incorporated in its entirety herein by reference. The covering layer is non-stick, e.g., siliconized or fluorinated at least on the side lying on the back layer.

The preparation of preferred transdermal therapeutic systems is performed by first mixing the constituents for the first layer, e.g. at first fentanyl and the gel former, dispersing (or dissolving parts of the fentanyl, respectively) in an organic medium such as heptane, and mixing the mineral oil and polyisobutylene in an organic medium, preferably the same as previously. Then, fentanyl and the gel former are dispersed in the mixture of polyisobutylene and mineral oil. In the production of this mixture preferably a volatile organic medium such as for example heptane is employed. Then, said mixture is applied as uniform layer onto the back layer and dried. If it is desired to apply a membrane this is applied on the side of the reservoir opposite to the back layer.

In a separate step the constituents of the second layer are mixed substantially in the same manner as described above for the first layer. However, in the second layer it is preferred that all of the fentanyl is brought into solution. The amount of fentanyl to be used should therefore be under the saturation solubility of the fentanyl in the second layer, and in the preparation it is to make sure that all of the fentanyl is dissolved.

Then, the mixture with the constituents of the second layer and the solvent is applied to the stripping film and dried.

Subsequently, the components obtained in both processing steps are laminated with each other in a way that the second layer is applied to the membrane, if such a membrane is provided. In the embodiments wherein no membrane is employed the second layer is laminated directly to the first layer. Thereafter, pieces of the desired size can be die-cut from the final laminated film and packaged.

In the individual processing steps the organic solvents required bring the polyisobutylene into solution and to disperse or dissolve the other constituents are usually removed by subjecting the products to increasing temperatures, optionally also using a low pressure.

With transdermal therapeutic systems according to the invention it is possible to provide practical patches for administering fentanyl or an analogue thereof that, over the known patches, in particular over the commercially available patches, contain less active ingredient and have a smaller patch area. In particular, with the administration of the active ingredient fentanyl a patch can be provided that is substantially bio-equivalent to the Durogesic products (see, definition in WO 02/074286), but contains significantly less active ingredient and is smaller. So, the DUROGESIC® SMAT patch providing a delivery rate of 25 μg/h contains 4.2 mg fentanyl, and the patch size is 10.5 cm². In comparison, a patch according to the invention also providing a delivery rate of 25 μg/h and being substantially bio-equivalent to the DUROGESIC® SMAT patch only contains 2.23 mg fentanyl, and the patch size is only 6.6 cm². These values are even better than the values of the polysilicone patches that are currently on the market, such as those of the product MATRIFEN® that with a delivery rate of 25 μg/h has only 2.75 mg fentanyl and a patch size of 8.4 cm².

The following examples explain the invention, but are not limiting.

### EXAMPLE 1 (COMPARATIVE EXAMPLE)

a) Preparation of the First Layer

At first, for the preparation of a transdermal patch fentanyl with an average particle size of 20 μm (e.g. Gesellschaft fur Mikronisierung mbH, jet mill, AS, Hosokawa Alpine AG) in heptane was dispersed with the polyisobutylene (mixture Oppanol B100:Oppanol B10 SFN 1:1, dissolved in n-heptane), silica (Cab-O-Sil M-5P) as the gel former, and mineral oil “Klearol” as the plasticizer. The mixture was applied as a thin layer to the back layer and dried so that an area weight of about 55 g/m² resulted.

b) Application of the Membrane

To this, a microporous polypropylene film (Celgard 2400) was applied as the membrane.

c) Preparation of the Second Layer

In parallel, only polyisobutylene (mixture Oppanol B100:Oppanol B10 SFN 1:1, dissolved in heptane) was applied as a thin layer to the stripping layer and dried so that an area weight of about 30 g/m² resulted. The tolerance of the area weights was 10%.

d) Preparation of the Patch

After the two layers were dried the two layers were laminated with each other, wherein the membrane was combined with the reservoir by using a suitable pressure.

Subsequently, rectangular patches with rounded edges with a size of 10 cm² were blanked and packaged with the covering film (patch size 10 cm²). The finished product had the following composition:

| | | |
|---|---|---|
| | | Concentration (%) of the |
| Name of the | | (dry) Reservoir ready for |
| Constituent | Amount | use, per Layer |

First Layer

| | | | |
|---|---|---|---|
| Fentanyl | 4.95 | mg | 9% |
| Mixture Oppanol B100: | 30.0 | mg | 54.5% |
| Oppanol B10 SFN 1:1 | | | |
| Cab-O-Sil M-5P | 0.55 | mg | 1% |
| Klearol (Mineral oil) | 19.5 | mg | 35.5% |

| | | |
|---|---|---|
| Heptane* | — | — |

Second Layer

| | | | |
|---|---|---|---|
| Mixture Oppanol B100: | 30.0 | mg | 100% |
| Oppanol B10 SFN 1:1 | | | |

| | | |
|---|---|---|
| Heptane* | — | — |

Layer Materials

| | | |
|---|---|---|
| Celgard 2400 | 10.0 | cm² |
| Hostaphan MN 19 MED | 10.0 | cm² |
| Primeliner 100 μm PET | 15 | cm² |
| C1S | | |
| PETP Film, 36 μm, | 15 | cm² |
| transparent | | |
| Aluminum Package, | 1 | sachet |
| white | | |

*not contained in the finished product

The components used in the example may be described in more detail as follows:

| Component Designation | Chemical Description | Function |
|---|---|---|
| Mixture Oppanol B100: | polyisobutylene adhesive | adhesive |
| Oppanol B10 SFN 1:1 | dissolved in n-heptane | |
| Cab-O-Sil M-5P | colloidal silica | gelling agent |
| Klearol | light mineral oil | plasticizer |
| Celgard 2400 | microporous polypropylene | membrane |
| | film | |
| Hostaphan MN 19 MED | polyethylene terephthalate | back layer |
| | film with a nominal | |
| | thickness of 19 μm | |
| Primeliner 100 μm | polyester film with a | stripping layer |
| PET C1S | nominal thickness of 100 μm, | |
| | one side siliconized | |
| PETP-Film, 36 μm, | polyester film with a | covering layer |
| transparent | nominal thickness of 36 μm, | |
| | one side siliconized | |
| Aluminum package, | heat-sealing package | packaging |
| white | | material |

### EXAMPLE 2

### According to the Invention

In the same manner as described in example 1 (comparative example) a transdermal therapeutic system was prepared by using the constituents as given in the following table with the concentrations given in said table. However, on the first layer no membrane has been applied and in the preparation of the second layer care has been taken that all of the fentanyl was completely dissolved. Accordingly, step b) of example 1 (comparative example) was not performed and in step d) the two layers were laminated with each other without covering the first layer with a membrane.

Subsequently, rectangular patches with rounded edges were blanked with a size of about 7 cm² and packaged with a covering film (nominal release 25 m/h, patch size 6.6 cm², total content of fentanyl 2.23 mg).

| | | Concentration (%) of the |
|---|---|---|
| | | (dry) reservoir ready for |
| Name of Constituent | Amount | use, per Layer |

First Layer

| Fentanyl | 1.98 | mg | 10% |
|---|---|---|---|
| Mixture Oppanol B100: | 10.59 | mg | 53.5% |
| Oppanol B10 SFN 1:1 | | | |
| n-Heptane* | — | | — |
| Silica | 0.20 | mg | 1% |
| (e.g. Cab-O-Sil M-5P) | | | |
| Light Mineral Oil (e.g. | 7.03 | mg | 35.5% |
| Klearol) | | | |

Second Layer

| | | | |
|---|---|---|---|
| Fentanyl | 0.25 | mg | 1.5% |
| Silica | 0.08 | mg | 0.5% |
| (e.g. Cab-O-Sil M-5P) | | | |
| Light Mineral Oil (e.g. | 0.83 | mg | 5% |
| Klearol) | | | |
| Oppanol B10 SFN | 13.81 | mg | 83.7% |
| Oppanol B100 | 1.54 | mg | 9.3% |

| | | |
|---|---|---|
| n-Heptane* | — | — |

Layer Materials

| | | |
|---|---|---|
| Scotchpak 9738 | 6.6 | cm² |
| Scotchpak 9742 | 11 | cm² |
| PETP Film, 36 μm, | 11 | cm² |
| transparent | | |
| Aluminum Package, | 1 | sachet |
| white | | |

*not contained in the finished product

### EXAMPLE 3

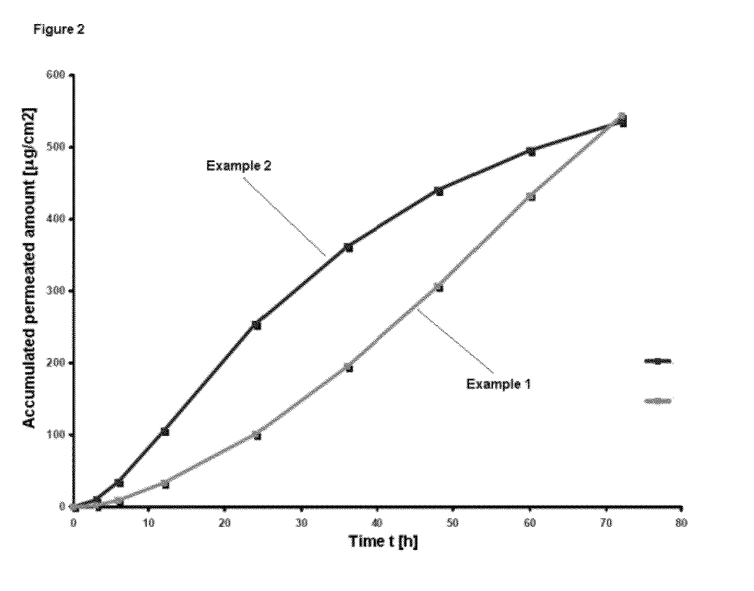

From the patches of example 1 (comparative example) and example 2 (example according to the invention) samples were blanked and the in vitro release profile of the fentanyl was determined on mouse skin in a common Franz cell. The result is shown in FIG. 2. Although the patch of example 1 has a higher active ingredient load than the patch of example 2 (0.495 mg/cm² over 0.338 mg/cm²) the in vitro release of the fentanyl from the patch according to the invention of example 2 is significantly faster than with the patch of comparative example 1.

### EXAMPLE 4

The patch of example 2 was compared in a clinical study in view of the in vivo blood level values in human patients achieved therewith with the commercial DUROGESIC® SMAT patch as reference patch. The commercial DUROGESIC® SMAT patch has an area of 10.5 cm² and an active ingredient load of 4.2 mg fentanyl. The TDS according to the invention has an area of 6.6 cm² at an active ingredient load of 2.23 mg fentanyl.

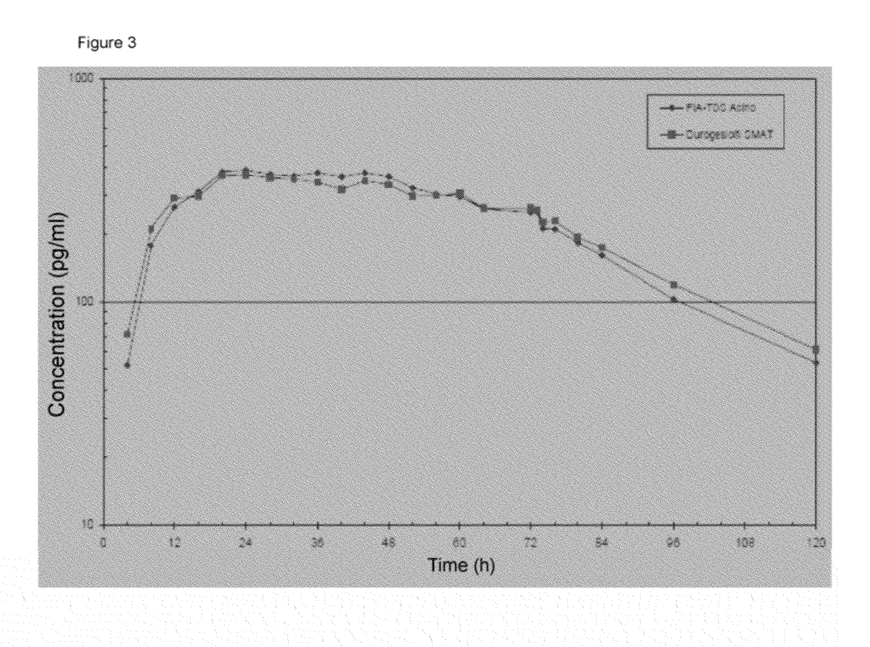

The result of the clinical study is shown in FIG. 3, the patch of example 2 is referred to as FIA-TDS Acino. It can be seen that the patch according to the invention causes a blood level of fentanyl comparable to that of the commercial reference patch, although the patch according to the invention is significantly smaller and contains less than half as much fentanyl as the commercial reference patch.

1. A transdermal therapeutic system for administering an active ingredient through the skin comprising:
a) a back layer,
b) a reservoir on the back layer comprising
b1) a first layer containing an active ingredient, at least one gel former, at least one plasticizer, and a first polyisobutylene; and
b2) a second layer containing an active ingredient, at least one gel former, at least one plasticizer, and a second polyisobutylene,
wherein the first polyisobutylene is different from the second polyisobutylene,
wherein at least the first layer contains undissolved active ingredient in the form of active ingredient particles; and
wherein the active ingredient in said first and second lavers is fentanyl or an analogue of the fentanyl.

2. The transdermal therapeutic system according to claim .1, wherein the first polyisobutylene comprises a mixture of (i) a polyisobutylene with a first average molecular weight and (ii) a polyisobutylene with a second average molecular weight, wherein the first average molecular weight is higher than the second average molecular weight.

3. The transdermal therapeutic system according to claim 1, wherein the second polyisobutylene comprises a mixture of (i) a polyisobutylene with a first average molecular weight and (ii) a polyisobutylene with a second average molecular weight, wherein the first average molecular weight is higher than the second average molecular weight.

4. The transdermal therapeutic system according to claim 3, wherein (1) the polyisobutylene with the first average molecular weight in the mixture of the first polyisobutylene has substantially the same average molecular weight as the polyisobutylene with the first average molecular weight in the mixture of the second polyisobutylene and (2) the polyisobutylene with the second average molecular weight in the mixture of the first polyisobutylene has substantially the same average molecular weight as the polyisobutylene with the second average molecular weight in the mixture of the second polyisobutylene.

5. The transdermal therapeutic system according to claim 2, wherein the polyisobutylene with the second average molecular weight has an average molecular weight in the range of 15,000 to 100,000 and the polyisobutylene with the first average molecular weight has an average molecular weight in the range of 500,000 to 10,000,000.

6. The transdermal therapeutic system according to claim 1, characterized in that the concentration of the active ingredient in the reservoir is in the range of 3% by weight to 20% by weight, based on the weight of all active ingredient-containing layers.

7. The transdermal therapeutic system according to claim 1, wherein the first layer is applied onto the back layer and contains the active ingredient in a higher concentration than the second layer.

8. The transdermal therapeutic system according to claim 1, wherein said system further comprises a stripping layer.

9. The transdermal therapeutic system according to claim 1, wherein either (i) the second layer is applied onto the first layer or (ii) membrane is applied onto the first layer and the second layer is applied onto the membrane.

10. The transdermal therapeutic system according to claim 1, wherein the second layer has a higher adhesive capacity than the first layer.

11. The transdermal therapeutic system according to claim 1, characterized in that the reservoir contains an amount of fentanyl or an analogue thereof sufficient to induce analgesia in a human patient and to maintain analgesia for a period of three to seven days.

12. The transdermal therapeutic system according to claim 1, characterized in that the plasticizer is a mineral oil and the gel former is colloidal silica or bentonite.

13. The transdermal therapeutic system according to claim 1, characterized in that the active ingredient particles in the reservoir are present in a micronized form with an average particle size of 50 mm or less.

14. The transdermal therapeutic system according to claim 1, characterized in that the back layer is an occlusive back layer.

15. The transdermal therapeutic system according to claim 1, characterized in that the first layer consists of 30 to 80% by weight of polyisobutylene and the second layer of 50 to 98% by weight of polyisobutylene, each based on the total weight of the respective layer.

16. The transdermal therapeutic system according to claim 1, characterized in that the plasticizer in the first layer is present in a concentration of 10 to 60% by weight and present in the second layer in a concentration of 1 to 15% by weight, each based on the total weight of the respective layer.

17. The transdermal therapeutic system according to claim 1, characterized in that the first layer contains the active ingredient in a concentration of 3% by weight to 15% by weight and the second layer contains the active ingredient in a concentration of 0.5% by weight to 5% by weight, each based on the total weight of all active ingredient-containing layers, wherein the concentration of the active ingredient in the first layer is higher than in the second layer.

18. The transdermal therapeutic system according to claim 1, characterized in that the active ingredient is selected from the group consisting of fentanyl, alfentanil, lofentanil, remifentanil, and sufentanil.

19. A method for the preparation of a transdermal therapeutic system according to claim 1, wherein (i) the constituents of the first layer and optionally one or more solvents are combined to form a mixture that is applied onto the back layer of the transdermal therapeutic system and the optional solvent, if present, is removed, (ii) the constituents of the second layer and optionally one or more solvents are combined to form a mixture that is applied onto a stripping layer and the optional if present, is removed, and (iii) a membrane is optionally applied onto the first or the second layer and the layers are laminated.

20. The transdermal therapeutic system according to claim 13, characterized in that the active ingredient particles in the reservoir are present in a micronized form with an average particle size of 20 mm or less.
